(19) United States
(12) Patent Application Publication
(10) Pub. No.: US 2015/0246162 A1
(43) Pub. Date: Sep. 3, 2015

(54) Biocompatible Implant

(71) Applicant: Adrian Robert Ford, Cardiff (GB)

(72) Inventor: Adrian Robert Ford, Cardiff (GB)

(21) Appl. No.: 14/633,651

(22) Filed: Feb. 27, 2015

(30) Foreign Application Priority Data
Feb. 28, 2014 (GB) 1403604.0

(51) Int. Cl. A61L 27/36; A61L 27/54; A61L 27/56

(52) U.S. Cl. CPC A61L 27/3654; A61L 27/3604; A61L 27/3687; A61L 27/56; A61L 27/54; A61L 2430/06; A61L 2420/02

(57) ABSTRACT

A biocompatible implant is disclosed for use in a patient, comprising a biological material having an open microporous structure; and a biologically active compound. The biologically active compound is impregnated within the open microporous structure of the biological material. The biologically active compound enhances cellular growth within the implant once inserted in the patient.

This invention relates to an implant, in particular a biocompatible implant to be used in partial meniscal replacement of the treatment of meniscal injuries.

The components of soft tissue, those tissues that exclude bones or teeth, are described widely in literature. The soft tissue is composed of two types of fibre. The first is collagen which is a fibrous protein providing tensile strength and the second is primarily proteoglyn which is composed of 2% protein and roughly 98% hyaluronic acid. The proteoglycan fibres have a limited amount of tensile strength. However they are highly hydroscopic and cause watery extra cellular fluid to form a gel like material. This “gel” helps oxygen and nutrients reach the cells within the tissue as well as helping to maintain the flexible and supple nature of the tissue.

It is well known to use surgical implants to replace or repair tissues in a human, in particular for the articulating surfaces of joints. This surgery is already common, but it is expected to be even more so given the ever aging population.

Articulating surfaces within a joint are composed of two hard surfaces fronted by soft connective tissues which are primarily there to provide lubricity, joint stability and to act as shock absorbing surfaces. One such area is the meniscus of the knee.

Each joint of the knee contains two menisci′ the medial meniscus which is located on the inner side of the joint and the lateral meniscus which is located on the outer side of the joint of the knee. Each meniscus is of a wedge shaped cross section and a semi lunar curvature. The outer wall of the meniscus is anchored to the joint capsule and via ligaments to the posterior and anterior aspects of the joint. The inner aspect and underlying surface of each meniscus is not anchored, allowing movement of the tissue as the femoral chondyles of the knee articulate across its surface.

As menisci of the knee are in high stress positions, injury in this region is a common occurrence in both humans and large animals. Such meniscal type tissue is also present in other joints.

Various efforts have been made to replace or repair damaged meniscus including total replacement of the meniscus with allograft, or partial replacement of the meniscus with an artificial material of a biological or non-biological composition. None of these options entirely and adequately meet the biological, surgical or practical demands.

For example, total allograft replacements are currently used to replace the entire meniscus in a patient and are anchored using a range of surgical techniques. The total replacement is restricted to patients who have undergone total menisectomy or who have no functional structure remaining. The scope of this procedure is limited by the availability of the material, the need to size match the donor tissue to the recipient, the challenging surgery and the mixed success rates of the surgery. Another potential drawback is that as the material donated is usually of cadaveric origin, they may hide pathogens with long or occult incubation periods, and as such may not be detectable using conventional testing techniques.

Partial Menisectomy replacements materials have been proposed which include both man-made and naturally occurring materials. While these devices are readily available and require little in the way of recipient matching, the downside is that the implant mechanical properties are not identical to the native tissue to which they are applied. As a result, date on the incorporation and repopulation of the material with the patient's cells, and hence longevity of the implant varies considerably.

The availability of tissue from a live donor is problematic because the patient match and size may delay the date of surgery, where on average it takes 3 m to find a match. Also it is common for implants to wear out after around 10-15 years. Whilst this duration may be suitable for a very elderly person, a younger person would be required to have two to three surgical procedures carried out over their lifetime, which would be inconvenient to the patient, since it seriously effects there quality of life and costly to the health service and or patient depending on the territory the surgery is provided.

It has also been found that the implants are prone to shrinkage, which again is detrimental to the lifetime of the implant since it contributed to the deterioration of the implant, leading to a further surgical procedure being required.

Whilst it is known to provide a biologically reactive coating to the implant, this does not encourage full integration of the implant with the recipients existing tissue.

Therefore, embodiments of the present invention are intended to address at least some of the above described problems and desires. In particular the implant is intended to ease implantation and fixation in acute or secondary menisectomy sites and to encourage cell generation between the tissue of the patient and the implant so as to promote acceptance and longevity of the implant in the patient, minimising the number of operations to be applied in the patient's lifetime. Also there is provided an implant that more closely matches the physical characteristics of the native tissue to which it is to be applied.

Therefore. improved physical properties of the implant are provided that enables it to be applied with the minimum amount of manipulation and increased availability.

According to a first aspect of the invention there is provided a biocompatible implant for use in a patient, comprising
a biological material having an open microporous structure; and
a biologically active compound wherein the biologically active compound is impregnated within the open microporous structure of the biological material such that the biologically active compound enhances cellular growth within the implant once inserted in the patient.

The biological material may be shaped prior to being used in a patient.

The biological material may be provided by a living donor who is not the patient. The biological material may be an allograft.

The biological material may have undergone a pre-treatment process causing plastic deformation of the biological material prior to use in a patient.

At least part of the biological material may further comprise a compound for providing hydrophilic properties to the implant.

The compound may be selected from the group comprising any one of, hyaluronic acid, glucuronic acid polymers, glucosamine polymers including glycosaminoglycans, kerata sulphate and chondroitin sulphate oligosaccharides, or a combination thereof.

At least part of the biological material may be hydroscopic.

The biologically active compound may be impregnated at a central region within the biological material. The central region is the region remote from the edges of the implant. The biological material may be fully impregnated with the biologically active compound.

The biologically active compound may be a compound for enhancing cellular ingress within the biological material.

The biologically active material may be selected from the group comprising glucuronic acid polymers, glucosamine polymers including glycosaminoglycans, kerata sulphate and chondroitin sulphate oligosaccharides, heparin, versican, brevican, neurocan, aggreca, catecholamines, adrenergic drugs, steroids and non-steroidal anti-inflammatories, cytokine and cytokine antagonists, and autologous or allogenic stem cells, hyaluronic acid, or a combination thereof.

The biologically active material may be hyaluronic acid comprising a molecular weight within the range of 3×10⁵ and 6×10⁵ kDa.

The biologically active material may be hyaluronic acid and may be provided at a concentration of 0.01 to 2 micro grams per ml.

The biological material may comprise a wedge shaped cross section.

A biocompatible implant according to any preceding claim, wherein the implant has a part annular structure when viewed from above.

The dimensions of the implant may be tailored to the patient to which it is to be applied prior to insertion of the implant within the patient.

In a further embodiment of the invention there is provided a method of forming a biocompatible implant, comprising the steps of:

treating the biological material so as to open the microporous structure of the biological material;

suspending the open microporous structure in a solution containing the biologically active compound so as to provide a mixture;

subjecting the mixture to a pressure or vacuum so as to impregnate the biological material within the microporous structure; and removing the impregnated biological material from the solution.

The pressure may be provided by positioning the biological material and the biologically active compound in a sealable receptacle, hermetically sealing the receptacle; and introducing carbon dioxide to within the sealed receptacle.

The solution may comprise an osmotically balanced or neutral solution and a biologically active agent.

Alternatively, the solution may be hypotonic or hypertonic.

The biologically active agent may be hyaluronic acid of a molecular weight of between 3-6×10⁵ kDA.

The biologically active compound may be hyaluronic acid and the concentration of the solution may be 0.6-1% w/v.

The pressure may be between −40 psi and +150 psi, preferably 50-70 psi and more preferably about 60 psi.

The mixture may be subjected to a pressure or vacuum whilst being at a temperature of between −5° C. and +30° C.

The pressure may be applied for a period of time between 0-72 hours.

The biological material either prior to or subsequent to the impregnation of the biologically active material is placed in a mould, applying a pressure or a vacuum through the mould for a period of time to deform the biological material to take the shape of the mould; and removing the biological material from the mould.

The biological material may be washed prior to the application of pressure or vacuum.

The method may further comprise applying the implant to a cryo-preservation solution and freezing the implant and the solution.

The method may further comprise transferring the freeze dried implant to a storage vessel.

The method may further comprise irradiating the implant.

The radiation dose may be between 0-25 kGy.

The implant may be irradiated with gamma radiation.

The implant may be irradiated with electrons.

In a further aspect of the invention there is provided a mould for forming an implant comprising a main support body having an internal moulding surface,

an opening located on the main support body for enabling access to the internal moulding surface;

the internal moulding surface having at least one pore; a lid co-operable with the opening of the main support body; and

a void region configured to enable the application of a vacuum or fluid under pressure.

The void region may be located below the moulding surface.

The shape and dimensions of the internal part of the mould may be dependent on the results of a 3D scan of the part of the recipient to be replaced or repaired.

In a further embodiment of the invention there is provided a biocompatible implant for use in a patient formed by

treating the biological material so as to open the microporous structure of the biological material;

suspending the open microporous structure in a solution containing the biologically active compound so as to provide a mixture;

subjecting the mixture to a pressure or vacuum so as to impregnate the biological material within the microporous structure; and removing the impregnated biological material from the solution.

Whilst the invention has been described above it extends to any inventive combination of the features set out above, or in the following description, drawings or claims. For example, any features described in relation to any one aspect of the invention is understood to be disclosed also in relation to any other aspect of the invention.

The invention will now be described, by way of example only, with reference to the accompanying drawings, in which:—

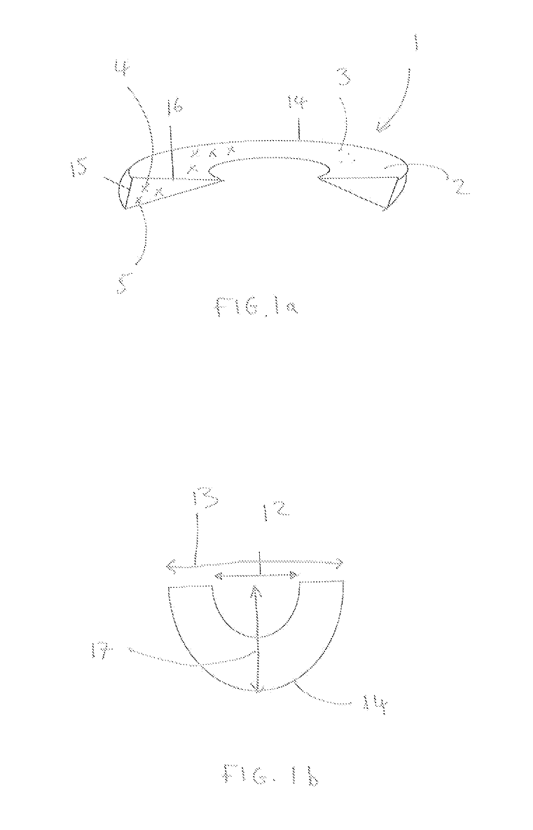

FIG. 1a is a perspective view of an implant according to the invention;

FIG. 1b is a top view of an implant according to the invention;

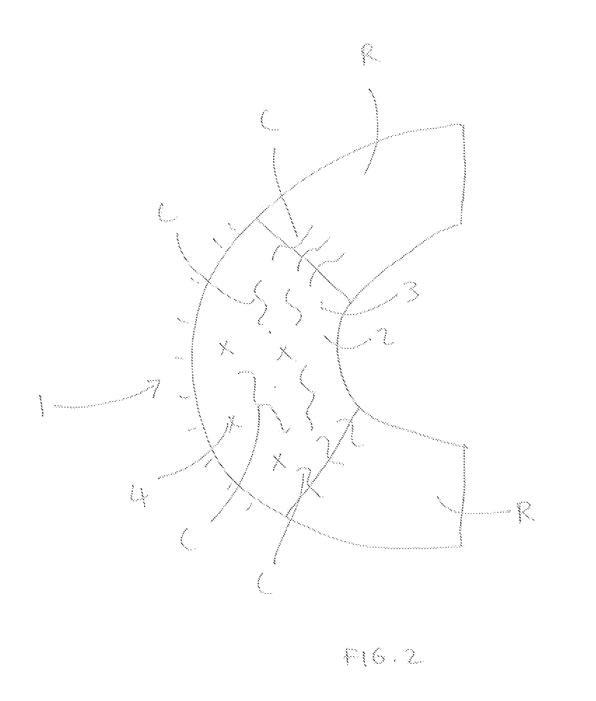

FIG. 2 is a schematic of an implant of FIG. 1 inserted in a meniscus of a recipient;

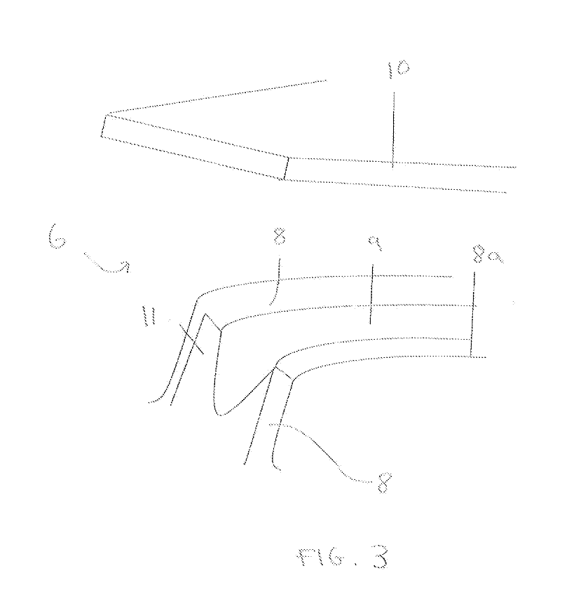

FIG. 3 is a schematic of the mould used to produce the final shape of the invention of FIG. 1; and

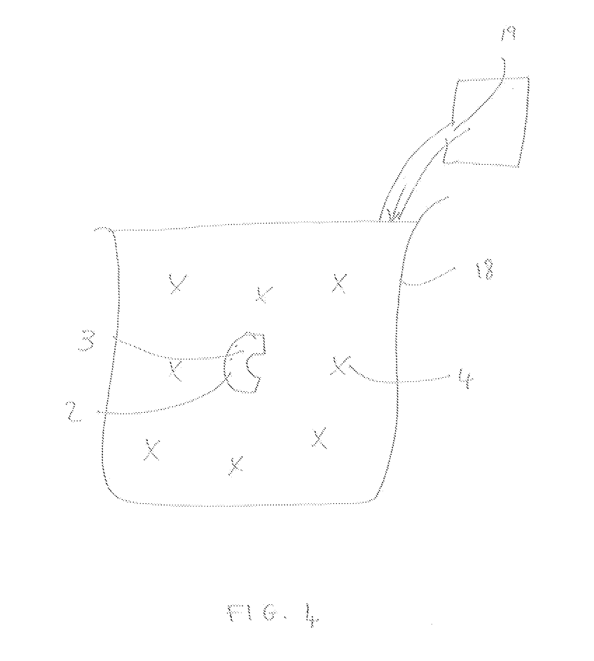

FIG. 4 is the starter material suspended in a solution containing the biologically active compound.

Referring firstly to FIGS. 1a and 1b, there is shown a biocompatible implant 1 for use in a patient, for example an implant for partial or total meniscal replacement. The biological material 2 has an open microporous structure 3. A biologically active compound 4 is impregnated within the open microporous structure 3 of the biological material 2. This arrangement enhances the cellular growth within the implant once inserted in the patient as shown in FIG. 2.

To produce the biocompatible implant 1, the biological material 2 undergoes physical manipulation which is undertaken in the presence of the biologically active compound 4 such that the residual concentration of the biologically active compound is at a biologically efficacious concentration within the implant 1.

During knee replacements the proximinal aspect of the tibia is removed along with the meniscal cartilages. In partial or uni compartmental replacements only one side of the joint surface and therefore one meniscus is removed. These tissues are removed from the bone and placed into isotonic saline at source. The material is then transferred to the tissue facilities where it is placed in cold storage, for example at a temperature of between 2°-8° C. while awaiting confirmation of necessary human tissue authority donor tests for pathogens and review of the donor consent forms and lifestyle questionnaires.

The meniscus is then removed from cold storage and anchoring ligaments typically found at the posterior and anterior aspects, along with any remnants of the capsular attachments are removed so as to provide an initial stage of reshaping of the material.

Therefore, the implant 1 is a biological implant and the starting material or biological material 2 is retrieved during partial or total knee replacements. As such, the biological material 2 is allograft derived from a living donor.

Therefore, it is possible to determine pathogens with a long or occult incubation period as the living donor may manifest the disease enabling the recipient to be informed and treated accordingly. Another benefit of using a biological starter material 2 of the implant 1 from a living donor is that the starting material closely matches or has approximately the same physical and structural properties of the native tissue. This minimises the rejection of the implant after insertion.

The biologically active compound 4 is used to enhance integration, lubricity and function of the implant 1. The biologically active compound is hyaluronic acid 5.

Other alternative suitable biologically active compounds 4 are glucuronic acid polymers, glucosamine polymers including glycosaminoglycans, kerata sulphate and chondroitin sulphate oligosaccharides, heparin, versican, brevican, neurocan, aggreca. The biologically active compound may also include agents to treat, reduce of mediate inflammatory or degenerative joint disease including catecholamines, adrenergic drugs, steroids and non-steroidal anti-inflammatories, cytokine and cytokine antagonists, and autologous or allogenic stem cells, or mixtures thereof.

The biological material 2 is washed and as shown in FIG. 3 is subsequently suspended in normal isotonic saline or a similarly osmotically balanced solution containing hyaluronic acid 5 of a molecular weight of between 3×10⁵ and 6×10⁵ kDa. The hyaluronic acid is therefore present within the body of the final implant at a concentration of 0.01-2 μg per ml or cubic centimetre, or 0.6-1% w/v. The mixture is then subjected to a pressure selected from a range of, for example, −40 psi to +150 psi. The preferred pressures are 50-70 psi, and more preferably about 60 psi. The vessel holding the material at the pressure range is held at a temperature of between 0 to 30° C. for a predetermined duration. The mixture is held at the preferred pressure for a period of time of between 0-72 hours.

Therefore, at the end of the treatment process the biologically active compound 4 is provided within the biologically active material 2 at a clinically significant concentration. This has been shown to have a beneficial effect on cellular recruitment and activity which is crucial to the repopulation and integration of the device into the patient's own tissue.

This pre-treatment opens the microporous structure 3 of the biological material 2 e.g. tissue, thereby altering the plastic deformation characteristics of the material and facilitating the impregnation of the material with the biologically active compound 4.

By applying a positive or negative pressure to the mixture, the biologically active compound 4 is inserted within the porous structure 3 of the biological material 2. Positive pressure may be provided by applying carbon dioxide into a sealed container containing the biological material 2 and the biologically active agent 4. Negative pressure may be provided by applying a vacuum.

The treatment of the starter material in this way ensures that the biologically active compound 4 penetrates deeply within the biological material 2. This then enhances the cellular ingress and therefore provides integration and activity of the implant 1 once it has been inserted in the patient. This provides an implant 1 having enhanced biological activity.

There is also provided a treatment of the biological material 2 to enhance the hydroscopic aspects of the material. The impregnation of the hyaluronic acid 5 within the biological material 2 increases the hydroscopic nature of the implant 1 both on the surface and within the body of the implant itself 1.

The preferred compound to be applied in the treatment is hyaluronic acid 5 as it is the most hydrophilic of the compounds found in tissue, but as an alternative any of the following may be used: glucuronic acid polymers, glucosamine polymers including glycosaminoglycans, kerata sulphate and chondroitin sulphate oligosaccharides.

It is important that the compound having hydrophilic properties penetrates the tissue and is not solely applied as a surface coating. The hydrophilic nature of the implant has a positive implication on cellular migration into the body of the implant. The implant is a graft based device. As a result this enhances migration of the patients own cells C into the device providing cell repopulation and integration of the device within the recipient R. It does not merely attempt to integrate the implant edge with the tissue so as to maintain the positioning of the implant 1. This is shown in FIG. 2.

The plastic deformation of the biological material 2 allows for a retrieved medial meniscal material to be used for medial or lateral applications.

To achieve the desired final shape, the biological material 2 is transferred to a mould carrier 6, the inside of which is based on the desired final shape of the implant 1. The implant 1 is then subjected to vacuum or pressure through the mould device 6 for a predetermined period of time until the necessary deformation and shape of the biological material 2 is achieved.

FIG. 3 shows that the moulds used to undertake the plastic deformation comprise a porous or perforated surface 7 to facilitate the application of a vacuum or CO₂ so as to vary the pressure applied to the biological material positioned in the mould. Therefore, the mould material is contained in a block or other form of support structure 6 to allow the application of negative pressure or positive pressure respectively.

The mould 6 further comprises open side walls 8 forming an internal moulding surface 9 and a lid 10 co-operable with the upper edge 8a of the side walls 8. The lid 10 can be removed to permit the biological material 2 to be placed within the mould 6 and re-application of the lid 10 hermetically seals the biological material 2 within the mould 6 enabling a positive or negative pressure to be applied. A void 11 is also provided where vacuum or fluid under pressure may be applied.

3D scanning technology is used to determine the required shape of the implant 1 to be applied to a recipient. The mould 6 is based on the results of the, for example, MRI scan so that the inner moulding surface 9 is customised depending on the scan results to form a donated graft material that matches the requirements of the recipient. As a result a customised biologically enhanced graft or implant 1 is achieved.

Positive and/or negative pressure may be applied in combination with low polarity hypotonic and hypertonic solutions to modify the plastic deformation characteristics of the material. The hypotonic and hypertonic solutions use simple sugars and salts. For example, the hypotonic solution comprises half normal saline 0.33% sodium chloride and 2.5% dextrose in water. The hypertonic solution however comprises 5% dextrose in lactated ringers solution.

Whilst sharp reshaping of the initial biological starter material is appropriate to provide an initial shape, it is the elastic deformation qualities of the biological material 2 that allow the final shape of the implant 1 to be provided by means of the pressure or vacuum application and the mould 6. It is the combination of washing, then applying a positive or negative pressure at a predetermined temperature so as to alter the nature of the plastic deformation of the material that achieves the final desired mechanical and physical nature of the implant device 1. This enables the implant 1 to be shaped to fit the recipient, whereby the shape of the final implant 1 allows for the distribution of load and provides a simple implantable device that requires the minimum amount of manipulation prior to fixation within the recipient.

The final implant 1 is shaped to provide a uniform wall, wedge shaped cross-section, uniform anterior and posterior ends and uniform underlying surface.

The shape of the implant provides sufficient depth to allow typical anchorage sutures or other surgical fixation devices (not shown) to be placed into the body of the implant 1 during implantation.

The shaping of the implant 1 provides an implantable device that requires a minimum amount of recipient size matching. The implant 1 is able to emulate a portion of the native meniscus. Also the implant 1 is designed such that the shape allows the implant 1 to sit correctly within the surgical area of resection or the area in which the implant is to be applied.

In the case of meniscal tissue, the shape allows lateral menisci to be deformed to match the requirements of a medial replacement. This deformation could be undertaken at any stage within the process. The final shape allows the outer wall of the device to abut the remaining capsular wall interface that is present following partial menisectomy or is generated as part of the implantation process for the device. This contact shall prove crucial in the longevity and incorporation of the meniscus.

The size of the implant 1 may depend on the recipient, but common sizes are as follows: the diameter of the interior curved edge of the annulus 12 has a length of between 14-44 mm; the diameter of the outer curved edge of the annulus 13 has a length of between 25-54 mm; the outer curved peripheral edge of the implant 14 has a length of between 65-110 mm; the thicker side edge of the wedge shaped cross section 15 has a thickness of between 6-13 mm; the distance of a sloped edge extending between the outer and inner peripheral side edges 16 has a length of between 25-40 mm and the bisecting line 17 of the part annular structure has a length of between 20-50 mm.

Once the final shape of the implant 1 has been provided, a final process is applied to the implant 1 to make it suitable for storage and transportation. For example, the biocompatible implant may be suspended in a cryo preservation solution, for example glycerol, and may be frozen at −80° C. The implant 1 may therefore be freeze dried and transferred to a final storage vessel.

The implant 1 may also be irradiated within the range of 0-25 kGy using gamma, or electron beam irradiation. Although, other irradiation techniques may be applied, i.e. using X-rays.

As shown in FIG. 4, the biologically active compound 4 may be applied to the biological material 2 in a standard vessel 18. The fluid mixture and the biological material 2 are then placed under pressure using carbon dioxide gas 18 applied via an inlet pipe 19. This causes impregnation of the biologically active compound 4. The moulding process which forms the desired shape of the implant 1 is applied prior to or after impregnation of the biologically active compound 4 in the biological material 2.

Alternatively, the biologically active compound 4, in the form of a fluid, is presented through the mould 6 itself via the pores 7 located in the inner surface of the mould 6. This enables the impregnation and the shaping to be carried out simultaneously thereby increasing the efficiency of implant 1 production. This also enables the production of the implant 1 to be an automated process.

Various modifications to the principles described above would suggest themselves to the skilled person. For example, other implant shapes may be provided depending on the application of the implant and the final shape of the implant may alternatively, or additionally be achieved via sharp resection.

The mould 6 may not comprise pores 7 and may instead comprise slots, filters or a mesh to facilitate the application of the vacuum.

Whilst it is described for the biological material 2 to be obtained from a living donor, it may instead be sourced from a deceased donor.

Whilst it has been described that the donor is a human, it is also envisaged that the donor may be an animal i.e. xenograft starter materials may be provided.

When impregnating the biologically reactive compound 4 within the biological material 2, the pressures may not be limited to the range of −40 psi to +150 psi and the duration for which the mixture is held at this pressure may vary.

In an alternative embodiment, the biological material may be suspended in a Isotonic, Hypotonic or Hypertonic solution which contains hyaluronic acid of a molecular weight of between 3-5×10⁵ and a concentration of 0.01-2 μg per ml or cubic centimetre, or 0.6-1% w/v. The mixture is then subjected to a range of pressures, for example −40 psi to +150 psi. It has been found that the plastic deformation of the material is enhanced via the use of solutions of low, medium and high osmolarity.

The vessel holding the material at the pressure range is held at a variety of temperatures ranging from 0 to 30° C. for a predetermined duration.

Whilst it is known for hyaluronic acid to have a limited effectiveness, the fact that it is impregnated into the body of the graft enables the effectiveness of the Hyaluronic acid to be longer than current applications where the hyaluronic acid is applied as a coating.

When required, the size of the implant may be cut to a defined size via sharp inception.

In summary, the implant is a recovered allograft which has been processed and pressurised in the presence of a biologically active component.

The allograft undergoes a process combining washes in solutions of different osmolarities, in a vacuum or high pressure environment to modify the plastic deformation characteristics of the material, followed by impregnation of the material with the biologically active compound to a clinically significant level within the body of the material. The biologically active component is hyaluronic acid which has been shown to have a positive effect on the cellular proliferation and activity especially in the repair and recovery of meniscal injuries.

By varying the nature of the process the mechanical and physical performance of the material can ease the manufacture of the final device and its physical performance. Further, the final dimensions of the material can be achieved either by sharp resection of the material, application of a vacuum or pressure used in combination with a suitable mould, or by combining these two techniques so as to achieve desired dimensions.

The sourcing, modifications and physical characteristics of the starter material from a live donor will increase implant availability, performance and increase the ease of implantation compared to currently available devices.

1. A biocompatible implant for use in a patient, comprising
a biological material having an open microporous structure; and a biologically active compound wherein the biologically active compound is impregnated within the open microporous structure of the biological material such that the biologically active compound enhances cellular growth within the implant once inserted in the patient.

2. A biocompatible implant according to claim 1, wherein the biological material is shaped prior to being used in a patient.

3. A biocompatible implant according to claim 1, wherein the biological material is an allograft provided by a living donor who is not the patient.

4. A biocompatible implant according to claim 1, wherein the biological material has undergone a pre-treatment process causing plastic deformation of the biological material prior to use in a patient.

5. A biocompatible implant according to claim 1, wherein at least part of the biological material further comprises a compound for providing hydrophilic properties to the implant.

6. A biocompatible implant according to claim 1, wherein the biologically active material is selected from the group comprising glucuronic acid polymers, glucosamine polymers including glycosaminoglycans, kerata sulphate and chondroitin sulphate oligosaccharides, heparin, versican, brevican, neurocan, aggreca, catecholamines, adrenergic drugs, steroids and non-steroidal anti-inflammatories, cytokine and cytokine antagonists, and autologous or allogenic stem cells, hyaluronic acid, or a combination thereof.

7. A biocompatible implant according to claim 1, wherein the biologically active compound is impregnated at a central region within the biological material.

8. A biocompatible implant according to claim 1, wherein the biologically active compound is a compound for enhancing cellular ingress within the biological material.

9. A biocompatible implant according to claim 1 wherein the biologically active material is hyaluronic acid comprising a molecular weight within the range of 3×10⁵ and 6×10⁵ kDa or is provided in a concentration of 0.01 to 2 micro grams per ml.

10. A biocompatible implant according to claim 1, wherein the biological material comprises a wedge shaped cross section.

11. A biocompatible implant according to claim 1, wherein the implant has a part annular structure when viewed from above.

12. A biocompatible implant according to claim 1, wherein the dimensions of the implant are tailored to the patient to which it is to be applied prior to insertion of the implant within the patient.

13. A method of forming a biocompatible implant of claim 1, comprising the steps of:
treating the biological material so as to open the microporous structure of the biological material;
suspending the open microporous structure in a solution containing the biologically active compound so as to provide a mixture;
subjecting the mixture to a pressure or vacuum so as to impregnate the biological material within the microporous structure; and
removing the impregnated biological material from the solution.

14. A method according to claim 13, wherein the pressure is provided by positioning the biological material and the biologically active compound in a sealable receptacle,
hermetically sealing the receptacle; and
introducing carbon dioxide or a vacuum to within the sealed receptacle.

15. A method according to claim 13, wherein the solution comprises an osmotically balanced or neutral solution, or hypotonic or hypertonic and a biologically active agent.

16. A method according to claim 13, wherein the biologically active agent is hyaluronic acid of a molecular weight of between 3-6×10⁵ kDa.

17. A method according to claim 13, wherein the pressure is between −40 psi and +150 psi, preferably 50-70 psi and more preferably about 60 psi.

18. A method according to claim 13, wherein the mixture is subjected to a pressure or vacuum whilst being at a temperature of between −5° C. and +30° C.

19. A method according to claim 13, wherein the pressure is applied for a period of time between 0-72 hours.

20. A method according to claim 13, further comprising placing the biological material in a mould either prior to or subsequent to the impregnation of the biologically active material;
applying a pressure or a vacuum through the mould for a period of time to deform the biological material to take the shape of the mould; and
removing the shaped biological material from the mould.

21. A method according to claim 13, further comprising applying the implant to a cryo-preservation solution and freezing the implant and the solution.

22. A method according to claim 13, further comprising irradiating the implant.

23. A mould for forming an implant according to claim 1 comprising a main support body having an internal moulding surface,
an opening located on the main support body for enabling access to the internal moulding surface;
the internal moulding surface having at least one pore; a lid co-operable with the opening of the main support body;
and a void region configured to enable the application of a vacuum or fluid under pressure.

24. A mould according to claim 23, wherein the shape and dimensions of the internal part of the mould are dependent on the results of a 3D scan of the part of the recipient to be replaced or repaired.

25. A biocompatible implant for use in a patient formed by treating the biological material so as to open the microporous structure of the biological material;
suspending the open microporous structure in a solution containing the biologically active compound so as to provide a mixture;
subjecting the mixture to a pressure or vacuum so as to impregnate the biological material within the microporous structure; and
removing the impregnated biological material from the solution.
